(19) United States
(12) Patent Application Publication
(10) Pub. No.: US 2025/0185938 A1
(43) Pub. Date: Jun. 12, 2025

(54) APPARATUSES, SYSTEMS, AND METHODS FOR USING MAGNETIC RESONANCE IMAGING TO ASSESS PERIODONTAL HEALTH

(71) Applicants: DENTSPLY SIRONA Inc., York, PA (US); SIEMENS HEALTHCARE GmbH, Erlangen (DE)

(72) Inventors: Kim BURZAN, Birkenau (DE); Johannes ULRICI, Darmstadt (DE); Lars LAUER, Neunkirchen (DE); Andreas GREISER, Erlangen (DE)

(73) Assignees: DENTSPLY SIRONA Inc., York, PA (US); SIEMENS HEALTHCARE GmbH, Erlangen (DE)

(21) Appl. No.: 18/531,834

(22) Filed: Dec. 7, 2023

(51) Int. Cl. A61B 5/055; A61B 5/00; G16H 50/30

(52) U.S. Cl. CPC A61B 5/055; A61B 5/742; G16H 50/30

(57) ABSTRACT

Apparatuses, systems, and methods are provided that use magnetic resonance imaging (MRI) to assess aspects of periodontal health of an individual. MRI data is obtained from a scan of an oral area of the individual. Analyzed MRI data is generated from the MRI data, wherein anatomical structures, anatomical abnormalities, and transition areas between anatomical structures/abnormalities are identified in the analyzed MRI data. Assessments relating periodontal health are made based on the analyzed MRI data. The assessments may be determinations of depths of dental pockets, inflammation of gum tissue, and/or distances that teeth are embedded in the bone. Assessment criticality scores may be determined from the assessments. The assessments, assessment criticality scores, and images rendered from the MRI data may be displayed on a display device.

### BACKGROUND

### Field of the Invention

Embodiments of the invention relate to apparatuses, systems, and methods that use magnetic resonance imaging (MRI) to assess aspects of periodontal health of an individual.

### Related Art

Periodontal health is a crucial part of maintaining overall oral health and well-being. Periodontal health refers to the health of the periodontium, which is the specialized tissues that surround and support the teeth, maintaining the teeth in the alveolar process of the maxillary and mandibular bones. Generally speaking, periodontal professionals evaluate gums, connective tissues, and the alveolar bone.

Dental pockets, also known as periodontal pockets or gum pockets, are spaces that form between the gum tissue and teeth. Periodontal charting or measurement of dental pockets describes a way of measuring the space between teeth and gum tissue. This is a standard diagnostic procedure used in assessing periodontal health. The pocket measurements are usually performed by a periodontal professional (e.g., general practitioner or dental hygienist) inserting a tip of a probe between the gum and the tooth to the bottom of the pocket. The probe has measurement markings such that the periodontal profession can determine the depth of the space between the gum and the tooth. This procedure is often long and painful for the patient. But the procedure provides important periodontal information. For example, periodontitis, which is a set of inflammatory conditions affecting gum tissue, can often be diagnosed based on increasing probe depth and bleeding when probing. Once the charting is complete, the individual's periodontal disease classification is calculated, based on the amount of bone loss according to national guidelines. This calculation gives the patient a grade of their periodontal disease, which can be used as basis for future monitoring of future monitoring of their condition. Jack G. Canton et al., A new classification scheme for periodontal and peri-implant diseases and conditions—Introduction and key changes from the 1999 classification, 45 J. of Clinical Periodontology (Supp. 20) S1 (2018).

Imaging technologies are commonly used for periodontal diagnostics, dental impression (DI) scans. For example, X-ray techniques such as cone beam computed tomography (CBCT) are used to assess tooth-supporting bone defects, as well as 2D intraoral or 2D extraoral X-ray techniques. While such standard periodontal imaging techniques can provide important diagnostic information, the imaging techniques are not well-suited to provide pocket depth and other periodontal diagnostic information. This is because the standard periodontal imaging techniques cannot provide detailed information about areas below the surface of gums. X-ray techniques, for example, cannot visualize soft-tissue processes such as inflammatory changes, which are linked to water retention in the bone. Thus, X-ray modalities do not show early changes within bone structures before inflammation-induced bone loss and cannot determine this at an early stage.

MRI is a non-invasive imaging technology that produces detailed images of anatomy and physiological processes. MRI is often used for disease detection, diagnosis, and treatment monitoring. The technology employs powerful magnets to produce a strong magnetic field that forces protons in a patient's body to align with the field. A radio frequency current is then pulsed through the patient to stimulate the protons, thereby causing the protons to spin out of equilibrium and strain against the pull of the magnetic field. After the radio frequency current is turned off, sensors can detect the energy released as the protons realign with the magnetic field. The time it takes for the protons to realign with the magnetic field and the amount of energy that is released change depend on the environment and the chemical nature of the molecules. Professionals can assess anatomy and physiological processes based on the resulting MRI data.

As compared to the standard periodontal imaging techniques, such as 2D radiographic techniques (intraoral periapical (PA) radiographs, bitewings, or extraoral panoramic X-rays) and three-dimensional cone beam computed tomography (CBCT), MRI provides better contrast in images of soft tissues. The possible use of MRI as a periodontal tool has been recognized. See Monika Probst et al., Magnetic Resonance Imaging as a Diagnostic Tool for Periodontal Disease: A Prospective Study with Correlation to Standard Clinical Findings—Is there added value?, 48 J. Clin. Periodontology 929 (2021); Maurice Ruetters et al., Dental magnetic resonance imaging for periodontal indication—a new approach of imaging residual periodontal bone support, 77 Acta Odontol Scand. 49 (2018).

### SUMMARY OF THE INVENTION

According to one embodiment of the invention, an apparatus provides an assessment related to an aspect of periodontal health of an individual. The apparatus comprises at least one processor configured to read out and execute instructions stored in at least one memory to thereby cause the apparatus to function as a plurality of units. One of the units is an MRI data receiving unit for receiving MRI data that was generated using an MRI scanning machine, the MRI data having been generated from a scan of an oral area of the individual, with the oral area including at least part of teeth, gum tissue, and a bone of the individual. Another of the units is an MRI analysis unit for generating analyzed MRI data wherein at least one of hard tissue, soft tissue, an anatomical abnormality, a transition area between two tissues, and a transition area between tissue and an anatomical abnormality is identified. At least one assessment unit is included for generating an assessment of the analyzed MRI data. And an output unit is included for outputting the assessment generated by the at least one assessment unit.

According to another embodiment of the invention, a method is provided for assessing an aspect of the periodontal health of an individual. The method comprises using an MRI scanning machine to scan an oral area of an individual to thereby generate MRI data. The MRI data is analyzed to thereby generate analyzed MRI data in which at least one of hard tissue, soft tissue, an anatomical abnormality, a transition area between two tissues, and a transition area between tissue and an anatomical abnormality is identified, with the analysis being performed by a computer. An assessment of periodontal heath of the individual is provided based on the assessment of the analyzed MRI data, with the assessment being provided by the computer.

According to yet another embodiment of the invention, there is provided a non-transitory computer readable storage medium storing one or more programs configured for execution by a computer having one or more processors and one or more memories. When the programs are executed by a computer, the computer functions as an MRI data receiving unit for receiving MRI data that was generated using an MRI scanning machine, the MRI data having been generated from a scan of an oral area of the individual, and the oral area including at least part of teeth, gum tissue, and a bone of the individual; an MRI analysis unit for generating analyzed MRI data in which at least one of hard tissue, soft tissue, a transition area between two tissues, and a transition area between tissue and an anatomical abnormality is identified; at least one assessment unit for generating an assessment relating to an aspect of periodontal health based on the analyzed MRI data; and an output unit for outputting the assessment generated by the at least one assessment unit.

### BRIEF DESCRIPTION OF THE DRAWINGS

FIG. 1 is a block diagram illustrating software components and hardware components of a system according to embodiments of the invention.

FIG. 2 shows analysis of an MRI image according to embodiments of the invention.

FIG. 3 is a flow chart of a method according to embodiments of the invention.

### DETAILED DESCRIPTION OF THE INVENTION

Embodiments of the invention will now be described. The embodiments include apparatuses, systems, and methods that use MRI to assess aspects of periodontal health of an individual.

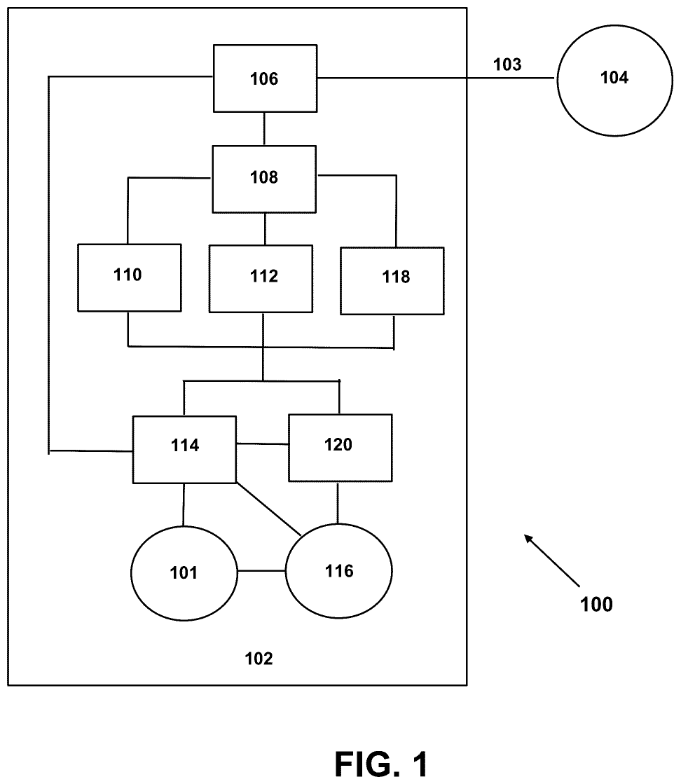

A system 100 according to embodiments of the invention is shown in FIG. 1. The system 100 includes a computer 102 and an MRI machine 104. As will be described below, the MRI machine 104 scans an individual and transfer the MRI data to the computer 102. The computer 102 is analyzes the MRI data and provides an assessment of the analyzed MRI data to a user of the system 100.

The MRI machine 104 is configurable scan oral areas of an individual. As used herein, an oral area means the area around a mouth of a human or other animal. The oral area may include teeth, gum tissue, and surrounding nerves and bone structures that relate to periodontal health. As used herein, an “anatomical structure” includes hard tissue such as teeth and bones and soft tissue such as gum tissue. Further, an “anatomical abnormality” is an abnormal anatomical structure, for example, inflamed tissue or a gap where an anatomical structure is usually present. The MRI machine 104 includes well-known structures to conduct MRI sequences, which are settings of pulse sequences and pulsed field gradients that result in particular image appearances. Those skilled in the art will appreciate how the MRI sequences may be optimized to generate MRI data conducive to the analyses and assessments as will be described below. As will also be appreciated by those skilled in the art, the MRI machine 104 could take a wide variety of forms, and the MRI scanning process may include a variety of techniques to produce results suited to different applications. For example, MRI contrast agents, such as gadolinium-based contrast agents (GBCAs), could be used to improve the visibility of structures targeted in the scan. As another example, when the system is used to assess aspects of periodontal health, the radiofrequency coil used in the system could be an intra-oral coil. Further, data resulting from a scan by the MRI machine comprises pixels and/or voxels that may be rendered to produce two-dimensional and/or three-dimensional images.

The computer 102 that receives the MRI data from the MRI machine 104 may be a general or special purpose computer. Such computers will typically include a central processing unit (CPU) that includes at least one processor. Such computers may in also included additional processing structures, such as graphical processor of a graphical processing unit (GPU). The one or more processors are coupled to one or more memory structures, which may be random access memory (RAM) devices, cache memories, non-volatile or backup memories, read-only memories, etc. In addition, memory may be considered to include memory storage physically located elsewhere in computer, e.g., any cache memory in a processor, as well as any storage capacity used as a virtual memory, e.g., as stored on a mass storage device (such as data storage device 116 described below) or on another computer coupled to the computer 102. It should be noted that a cloud-based architecture could also be used for system 100. As will be appreciated by those skilled in the art, cloud computing is the delivery of services—such as servers, storage, databases, networking, software, analytics, and intelligence—over the internet (“the cloud”). As an example of a cloud computing embodiment of the invention, a server computer could receive the MRI data from the MRI machine 104 and the computer 102 could be client that accesses the MRI data from the server.

The computer 102 will also typically include at least one communication interface comprising a number of input and output components for communicating with external devices. Such a communication interface can be provided with a wide variety of technologies. For example, the communication interface components may be operable to couple the computer 102 to a network or devices. For example, the communication interface may include wired communication components, wireless communication components, cellular communication components, Near Field Communication (NFC) components, BLUETOOTH® components, WIFI® components, and other communication components to provide communication via other modalities. In embodiments of the invention, input and/or output components of the communication interface provides for communication to the MRI machine 104 through the operative link 103, as will be described below.

The computer 102 further includes a data storage device 116. The data storage device 116 is configured to record data persistently, where “persistently” or “persistent” refers as to a device's ability to maintain recorded data after loss of power. In some embodiments, the data storage device 116 may correspond to non-disk storage media. For example, the data storage device 116 may be one or more solid-state drives (SSDs), flash memory-based storage, any type of solid-state non-volatile memory, or any other type of non-mechanical storage device. In other implementations, the data storage device 116 may include mechanical or spinning hard disk, such as hard-disk drives (HDD). As noted above, the computer 102 could also be used in a cloud-based system and thereby access stored data from a remote computer.

As an alternative to including the data storage device 116, or in addition to the data storage device 116, in embodiments, the computer 102 is operatively linked to a data storage device that physically separated from the structure of computer 102. For example, an input/output component of the communication interface of the computer 102 may be connected to a network through which the computer 102 receives data from an external data storage device.

The computer 102 includes a user interface 101 incorporating one or more user input devices (e.g., a keyboard, a mouse, a trackball, a joystick, a touch pad, and/or a microphone, among others) and a display device (e.g., an LCD display panel). The user interface 101 allows a user to control operation of the computer and view analyses and assessments of MRI data, as described herein. In other embodiments, the computer 102 is operatively linked to another device providing such a user interface. For example, the computer 102 may be connected to a network to which a remote workstation with a user interface is provided.

It should be appreciated that the computer 102 typically includes suitable analog and/or digital interfaces between processors and components as is well known in the art. Other hardware environments are contemplated within the context of embodiments of the invention.

The computer 102 operates under the control of an operating system and executes or otherwise relies upon various computer software applications, components, programs, objects, units, data structures, etc., as will be described in greater detail below. Moreover, various applications, components, programs, objects, units, etc. may also execute on one or more processors in another computer coupled to computer 102 via a network, e.g., in a distributed or client-server computing environment, whereby the processing required to implement the functions of a computer program may be allocated to multiple computers over a network.

The computer 102 may include a computer aided diagnostic program to implement one or more of the steps of the processes described herein. For the purposes of implementing such steps, an image database, storing medical image scans, may be implemented in computer 102 in the data storage device 116 or in another data storage device that is operatively linked to the computer 102, as described above. It will be appreciated, however, that some steps in the processes described herein may be performed manually and with or without the use of the computer 102.

The operative link 103 provided between the computer 102 and the MRI machine 104 allows the computer 102 to receive MRI data from the MRI machine 104. In further embodiments, the computer 102 includes a software unit or units enabling the user to control operation of the MRI machine 104 through the operative link 103. The MRI machine 104 and the computer 102 may be formed with a direct physical connection such that the operative link 103 is in the form of a wire structure that transmits the MRI image data and/or control commands between the computer 102 and the MRI machine 104. In other embodiments, the MRI machine 104 and the computer 102 are connected through a network, such as local area network (LAN), a wireless local area network (WLAN), or through the Internet. Those skilled in the art will appreciate the wide variety of ways that the MRI machine 104 and the computer 102 may be operatively linked.

In still other embodiments, the MRI machine 104 and the computer 102 are provide as a singular hardware structure. That is, the computer 102 is directly built into the MRI machine 104. Thus, the operative link between the computer 102 and the MRI machine 104 is formed by components of the singular structure.

It should be noted that in still further embodiments of the invention, the MRI data to be analyzed by the computer 102 is not received directly from an MRI scanning machine. For example, the MRI data may be generated using an MRI scanning machine, and the MRI data may then be saved in an external storage device linked to the computer 102 (e.g., through a network, as described above) or in the data storage device 116. Thus, at any time following the MRI scan that generates the MRI data, the computer 102 may receive the previously generated MRI data from the external storage device or from the data storage device 116.

As shown in FIG. 1, embodiments of the invention include functional units. The functional units reside in memory device(s) of the computer 102. Each of the units is computer-executable code that, when executed by the computer's processors, imparts the unit's functionality to the computer 102. Those skilled in the art will easily recognize the coding languages and techniques that may be used to create the units.

An MRI data receiving unit 106 receives the MRI data that is received from the MRI machine 104. That is, the MRI image data receiving unit receives operates with a communication interface of the computer 102 that that is connected to the operative link 103 between the computer 102 and the MRI machine 104.

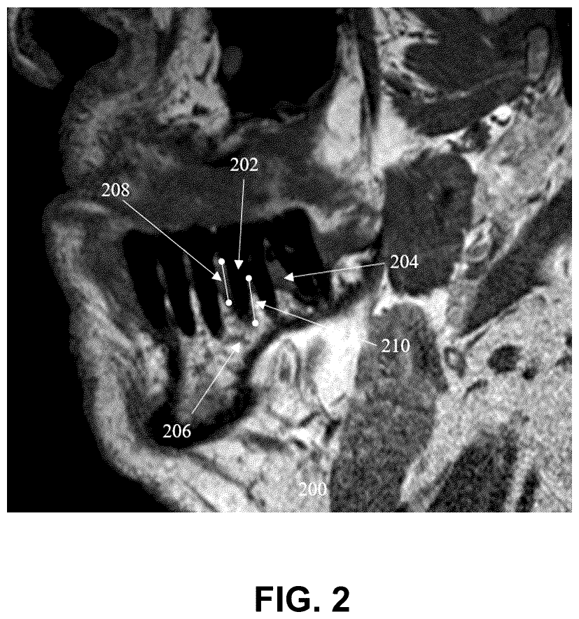

An MRI analysis unit 108 is provided to analyze the received MRI data receiving unit 106 to determine anatomical structure(s), including hard and soft tissue, anatomical abnormalities, transition area(s) between two tissues, and/or transition area(s) between tissue and anatomical abnormalities. The analysis performed by the MRI analysis unit 108 may take many different forms. In embodiments of the invention, the MRI analysis unit 108 is configured to analyze pixels or voxels in the MRI image data based on gray values of the pixels/voxels. Such MRI analysis based on gray-values of pixels/voxels is a known technique in the art. To illustrate such an analysis process, FIG. 2 shows a two-dimensional image 200 rendered from MRI data of a human's oral area. In the MRI image 200, a tooth 202 corresponds to a black area, gum tissue 204 corresponds to an intermediate gray area, and bone 206 corresponds to a white area. As the tooth 202, gum tissue 204, and bone 206 have distinct gray values, the MRI analysis unit 108 can determine these anatomical structures, as well as transition areas between the anatomical structures based on of the gray values, thereby creating analyzed MRI data. And the analysis process can be conducted throughout the oral area shown in FIG. 2. To further facilitate the analysis, the data from more than one MRI image may be used. More specifically, the MRI analysis unit 108 will designate areas in the MRI data where the gray values are substantially black as teeth, designate areas where the gray values are intermediate grays as gum tissue, and designate areas where the gray values are substantially white as bone. Note that a range of gray values may be designated to correspond to teeth, gum tissue, and bone.

After the image 200 is analyzed by the MRI analysis unit 108, a pocket depth assessment unit 110 is configured to automatically determine the pocket depths for the teeth. As shown in FIG. 2, the pocket depth assessment unit 100 can determine a line 208 that extends from an area at the top of the gum tissue 204 where the tooth 202 emerges from the gum tissue 204 to an area where the root of the tooth 202 enters the bone 206. The line 208 therefore represents the length of pocket depth adjacent to the tooth 202. Note that while the pocket depth measurement length 208 is shown along one tooth in FIG. 2, the process can be repeated for all of the pockets formed between the gum tissue and teeth in the oral area of MRI data. The length of the line 208 is calculated based on the known size of the pixels that are along the line. For example, each pixel might represent 0.5 mm (or other length), with the total length of the line 208 being the sum of the length of all the 0.5 mm pixels that lie along the line.

It should be noted that while analysis of gray values of pixels in a two-dimensional image 200 is depicted in FIG. 2 for ease of illustration, in embodiments of the invention the MRI analysis unit functions to analyze three-dimensional MRI data. In such cases, the gray values of voxels are analyzed to determine the anatomical structures, anatomical abnormalities, and transition areas shown in an oral area in the same manner as the gray values of pixels are analyzed in a two-dimensional image. Lines along teeth from the areas at the top of the gum tissue to the bottom areas where the tooth enters the bone are determined in the three-dimensional space defined by the voxels, and the lengths of the lines are calculated based on the sum of the known lengths of the voxels that lie along the lines. The general process of generating analyzed MRI data and subsequent assessment of the analyzed MRI data (e.g., pocket depth determination) is the same regardless of the number of images used and regardless of whether two-dimensional or three-dimensional MRI image data is used for the analysis.

By using the pocket depth assessment unit 110, a periodontal professional can obtain pocket depth information without performing a traditional pocket depth measurement process. As discussed above, the traditional process is often long and painful for the patient. Thus, the ability to obtain pocket depth assessments according to embodiments of the invention is a tremendous advance over the prior art.

In some embodiments of the invention, a manual measure unit (not depicted in FIG. 1) provides a manual measurement tool for the user interface 101 of the computer system 100. The manual measurement unit allows the user to draw a line on an image rendered from the MRI data shown on a display of the user interface 101, with the manual measurement unit thereafter calculating the length of the line in the same manner that the lengths of the automatically determined pocket length lines are calculated from known pixel/voxel sizes by the pocket depth assessment unit. The user could thereby use the measurement tool to measure distances in any part of an image rendered from the MRI data.

The computer system 100 also includes an inflammation assessment unit 112 configured to determine inflammation of gum tissue from the MRI data. The inflammation assessment unit 112 determines inflammation areas using the gray values of the pixels/voxels in the gum areas of the analyzed MRI data. Variations in the gray values in the gum areas can be correlated to assessment standards. For example, brighter gray values may be indicative of water-retention, and, thus, significant inflammation. The inflammation assessment unit 112 can thereby be configured to indicate that areas of the gums having gray values of a certain higher brightness have significant inflammation, areas of the gums having intermediate gray values have intermediate inflammation, and areas of the gums having darker gray values have little or no inflammation. Also using the brightness/darkness of the gray values, the inflammation assessment unit 112 can measure the size of the inflammation areas and provide a relative assessment of the amount of inflammation, for example, by comparing the size of the area with significant inflammation to the total area of the gum. The inflammation assessment unit 112 could provide further assessments of the gum based on the brightness/darkness of the gray values, such as an extent of scarring.

Still further, the inflammation assessment unit 112 may determine whether an inflammation is acute or chronic based on the combination of contrasts from T1-weighted and T2-weighted MRI sequences. As will be appreciated by those skilled in the art, T1-weighted and T2-weighted MRI sequences are commonly used in MRI scans. In the case of spin-echo pulsed sequences, for example, T1-weighed images are typically produced by using short time echo (TE) and repetition (TR) times, with the contrast and brightness of images thereby predominately determined by T1 properties. T2-weighed images are typically produced by using longer TE and TR times, with the contrast and brightness of the images thereby predominately determined by T2 properties. Whether inflammation is acute or chronic could be determined, for example, with acute changes being highlighted as signal increase in the T2 weighted images, whereas remodeled or scar tissue in the chronic state may only show signal changes in the T1 weighted images due to remaining structural changes. In one embodiment, the classification of pixels representing the condition of inflammation may be identified based on quantitative methods as already established for other applications (e.g., T1 Mapping based on MP2Rage or T2 mapping based on “GRAPPATINI” in neuro imaging, MyoMaps in Cardiac Imaging, MR Fingerprinting). The quantification of MRI parameters like T1 and T2 enables a more accurate and reproducible classification of tissue than measuring the contrast or signal changes in conventional single contrast image MRI sequences. For example, by measuring the pixel value at various suitable echo times, the apparent T2 relaxation curve of the pixel can be quantified. This T2 time is then ideally independent from any particular sequence or scanner property. Normal ranges for T1-weighted and T2-weighted scans may be established to define the threshold for classifying a pixel, e.g., as inflammatory. As already done for other MR indications, the quantification of T1 and/or T2 enables a differential diagnosis of the tissue status, e.g., classification as scar, fat, inflammation, and/or remote (healthy) tissue, even if at the expense of longer scans due to the need for sampling the same pixel with varied scan parameters. The classification of tissue based on such quantitative mapping is also less user-dependent.

The computer 102 further includes a teeth embedded assessment unit 118 for measuring distances that teeth are embedded into the corresponding alveolar bone process. That is, the teeth embedded distance unit 118 measures the distance 210 shown in FIG. 2, which extends from the area where the tooth 210 enters the bone 206 to the bottom of the root of the tooth 210 in the cortical bone 206. The teeth embedded assessment unit 118 can provide such measurements for all of the teeth in the analyzed MRI data. Measurements of the teeth embedded distances cannot be determined by any standard mechanical technique known in the art. Thus, the teeth embedded assessment unit 118 provides a significant advance over the prior art.

It should be noted that the assessment unit or assessment units provided with embodiments of the invention may be configured to provide both “local” assessments of one or a small set of anatomical structures, anatomical abnormalities, and transition areas in an oral cavity and “global” assessments wherein anatomical structures, anatomical abnormalities, and transition areas throughout the oral cavity are assessed. Further, embodiments of the invention may use anatomical information derived from the analysis of MRI data. For example, a landmark detection algorithm may first detect teeth long axes, such as by identifying roots and crown of a distinct tooth, and use the axes detection for characterizing the teeth embedded distance by evaluating signal variations around the tooth as a function of the position along the tooth's long axis.

An assessment scoring unit 120 is included in embodiments of the invention to provide scores of assessments determined by the assessment units.

In the case of pocket depth determinations, the assessment scoring units 120 determines one or more scores based on a comparison of a current pocket depth assessment of a patient with previous pocket depth assessment of the same patient. In such embodiments, the assessment scoring unit 120 can access the patient's previous pocket depth assessment from the data storage 116 or through a connection to a remote storage that is provided separate from the computer 102 as described above. The pocket depth assessment score could be output as a numerical value on a defined scale such as 1 to 10, wherein as score of 1 indicates little or no pocket depth changes for the patient, and a score of 10 indicates large pocket depth increases. In addition, or in alternative, the pocket depth assessment score could be provided as a color that is in relation to a color scale. For example, a green color could be used to indicate a low probability of periodontal problems based on the pocket depth assessment, a yellow color could be used to indicate a medium probability of periodontal problems based on the pocket depth assessment, and a red color could be used to indicate a high probability of periodontal problems based on the pocket depth assessment. The pocket depth assessment score and/or colors could be output by the output unit 114 (described below) for the display of the user interface 101.

In the case of inflammation assessment, the assessments scoring unit 120 determines one or more scores based on a comparison of a current inflammation assessment of a patient with a previous inflammation assessment of the same patient. In this regard, the assessment scoring unit 120 may access a previous inflammation assessment of the patient in the same way that it accesses a previous pocket depth assessment of the patient. Alternatively, the inflammation assessment score can be based on the quantified assessment of the analyzed MRI data. For example, the inflammation assessment score could be determined by the sizes or brightness of inflammation areas of the analyzed MRI data. As with the pocket depth assessment score, the inflammation assessment score could be provided as a number or color that is relative to a defined scale. For example, a green color could be used to indicate a low probability of periodontal problems based on the inflammation assessment, a yellow color could be used to indicate a medium probability of periodontal problems based on the inflammation assessment, and a red color could be used to indicate a high probability of periodontal problems based on the inflammation assessment. The inflammation assessment score and/or colors could be output by the output unit 114 for the display of the user interface 101.

In the case of the teeth embedded assessment, the score can be based on a comparison of a current teeth embedded assessment of a patient with previous teeth embedded assessment of the same patient. In this regard, the assessment scoring unit 120 may access a previous inflammation assessment of the patient in the same way that it accesses a previous pocket depth assessment and/or inflammation assessment of the patient. The teeth embedded determination score could be output as a numerical value on a defined scale such as 1 to 10, wherein as score of 1 indicates little or no teeth embedment changes for the patient, and a score of 10 indicates large reductions in the teeth embedment. In addition, or in alternative, the score could be provided as a color in relation to a defined scale. For example, a green color could be used to indicate a low probability of periodontal problems based on a finding that the tooth embedded distances have remained relatively constant over a period of time, a yellow color could be used to indicate a medium probability of periodontal problems based on a finding that the tooth embedded distances have moderately changed over a period of time, and a red color could be used to indicate a high probability of periodontal problems based on the tooth embedded distances deteriorating over a period of time. The teeth embedded assessment score and/or colors could be output by the output unit 114 for the display of the user interface 101.

In some embodiments, information from a longitudinal assessment of inflammation scores or pocket depth determinations may be visualized in an advantageous way to inform the patient on the success of the periodontitis treatment program. In particular, a visualization may be used as a motivation for carefully following dental care procedures as recommended by the dentist or even be fed into a reward system where the patient gets renumeration for improvements in the inflammation or pocket depth score.

The assessments scoring unit 120 may provide still further assessment scores and other information. For example, the assessments scoring unit 120 could provide an overall score indicative of periodontal health, with the score taking into account a combination of a pocket depth assessment, an inflammation assessment, and a tooth embedded assessment. Such an overall score could also take into account other factors determined using the system 100 or that are provided to the system. In this regard, the overall score may take into account factors such as a patient's age, lifestyle, periodontal hygiene, etc. Such additional factors could be entered into the system by a user and/or provided to the system from a data storage, e.g., a patient management system. Still further, the assessments scoring unit 120 can be configured to provide treatment suggestions based on the assessments made by the unit. For example, the assessments scoring unit 120 could provide a suggestion for an amount of time until a follow-up evaluation of a patient based on the current and past assessments.

The assessments from the assessments scoring unit 120 may be saved either locally, e.g., in the data storage device 116, or in a remote storage device, e.g., as part of a patient management system.

An output unit 114 is provided to output the pocket depth assessments made by the pocket depth assessment unit 110, the inflammation assessments made by the inflammation assessment unit 112, the teeth embedded distances determined by the teeth embedded assessments unit 116, and other assessments, determinations, calculations, analysis, etc. made by parts of the system 100. In the configuration depicted in FIG. 1, the output unit 114 sends its output to the user interface 101. In addition, or in alternative, the output unit can provide its output to the data storage 116 of the computer 102.

The output unit 114 can also output the MRI data received from the MRI scanning machine to allow for images from the MRI data to be rendered on a display device of the user interface 101 or stored in the data storage device 116. In further embodiments, the output unit 114 can be configured to provide its output to other systems outside of the computer 102 for display and/or storage. For example, the output unit 114 can be configured to send its output to a patient management system.

The display device provided with the user interface 101 may be configured to display all of the output from the output unit 114. That is, assessments made by the assessment units, scores made by the assessment scoring unit, and other assessments, determinations, calculations, analysis, etc. made by parts of the system 100 can be shown on the display device. The display device may also be configured to display 2D or 3D images rendered from the MRI data received from the MRI machine 104. The displayed images will provide a professional with details of dental pulp, nerves, gums, bone structure, and other useful periodontal information. The professional can thereby evaluate aspects of the individual's periodontal health, such as shape of bone, bone loss, extent of periodontal attachment, risk of tooth loss, etc.

The display may be further configured to display composite images that include one or more images rendered from the MRI data and images rendered from other imaging techniques. For example, the display may provide a composite image that includes one or more MRI images and one or more images obtained from a dental impression (DI) scanner. The composite image can be provided to the display of the user interface by the output unit 114, with the output unit 114 generating the MRI images from the MRI data received by the MRI receiving unit 106 and the output unit 114 obtaining the DI images from the data storage device 116 or another device operatively linked to the computer 102. Those skilled in the art will recognize the many techniques that could be used to form such composite images.

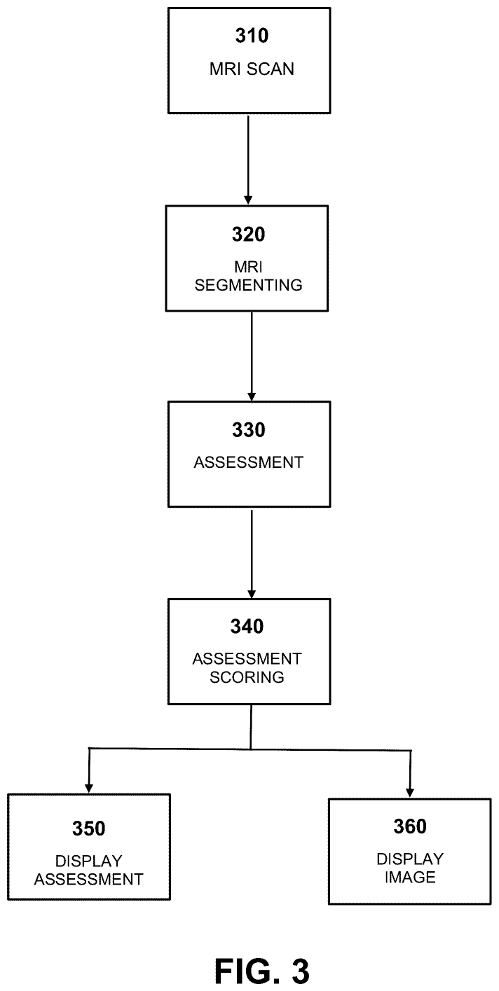

FIG. 3 is a flow chart of a method using MRI data to assess periodontal health according to embodiments of the invention. The method may be performed using a system according to the embodiments described above or with other systems according to embodiments of the invention.

The method begins with a step 310 of conducting an MRI scan of an oral area of an individual. The oral area can include teeth, gum tissue, and surrounding nerves and bone structures that relate to periodontal health. In many cases, the oral area includes all of the teeth, gum areas, cortical bones in which the teeth are embedded. The MRI scan thereby generate MRI data that can be used to render images of the scanned oral area and can be used to provide assessments of the periodontal health of the individual.

In step 320, the MRI data is analyzed so as to determine anatomical structure, anatomical abnormalities, and/or transition areas. As discussed above, the analyzed MRI data is generated using different gray values of pixels/voxels that make up the MRI data from a scan of the individual. In step 330 an assessment relating to the periodontal heath of the individual is generated based on the analyzed MRI data. The assessment may be, for example, calculated pocket depth determinations, determinations as to inflammation of the gum tissue, and/or calculated distances that teeth are embedded into cortical bone, as described above. In step 340, one or more assessment scores are determined as described above. In further step 350, the assessments and/or assessment scores are displayed on a display device. The displayed assessments and/or assessments scores may be presented in the form of numbers (e.g., calculated distances), relative numbers (e.g., a 1 to 10 scale), or colors, as described above. In step 360, which may be performed before, with, or after step 350, at least one MRI image and/or composite image that includes an MRI image component that is based on the MRI data is rendered on a display device.

Further aspects of the systems and methods described above may be implemented as an article of manufacture such as a non-transitory computer readable storage medium. The non-transitory computer readable storage medium may be readable by a computer and may comprise instructions for causing the computer to perform functions described herein. The non-transitory computer readable storage medium may be implemented by a volatile computer memory, non-volatile computer memory/storage, hard drive, solid-state memory, flash drive, removable disk and/or other media.

The terminology used in the description of the invention herein is for the purpose of describing particular implementations only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations described herein were chosen and described in order to best explain the principles of embodiments of the invention and its practical applications, to thereby enable others skilled in the art to best utilize embodiments of the invention and various implementations with various modifications as are suited to the particular use contemplated.

1. An apparatus for providing an assessment related to an aspect of periodontal health of an individual, the apparatus comprising:
at least one processor configured to read out and execute instructions stored in at least one memory to thereby cause the apparatus to function as:
an MRI data receiving unit for receiving MRI data that was generated using an MRI scanning machine, the MRI data having been generated from a scan of an oral area of the individual, and the oral area including at least part of teeth, gum tissue, and a bone of the individual;
an MRI analysis unit for generating analyzed MRI data in which at least one of hard tissue, soft tissue, an anatomical abnormality, a transition area between two tissues, and a transition area between tissue and an anatomical abnormality is identified; and
at least one assessment unit for generating an assessment relating to an aspect of periodontal health of the individual based on the analyzed MRI data; and
an output unit for outputting the assessment generated by the at least one assessment unit.

2. An apparatus according to claim 1, wherein the at least one assessment unit includes a pocket depth assessment unit that determines, from the analyzed MRI data, depths of dental pockets that extend from areas at a top of the gum tissue to areas where the teeth enter the bone.

3. An apparatus according to claim 1, wherein the at least one assessment unit includes an inflammation assessment unit that determines, from the analyzed MRI data, inflammation of the gum tissue.

4. An apparatus according to claim 1, wherein the at least one assessment unit includes (i) pocket depth assessment unit that determines, from the analyzed MRI data, depths of dental pockets that extend from areas at a top of the gum tissue to areas where the teeth enter the bone, and (ii) an inflammation assessment unit that determines, from the analyzed MRI data, inflammation of the gum tissue.

5. An apparatus according to claim 4, wherein the at least one assessment unit includes an inflammation assessment unit that determines, from the analyzed MRI data, inflammation of the gum tissue and whether the inflammation is acute or chronic.

6. An apparatus according to claim 5, wherein the MRI data includes data from T1-weighted and T2-weighted scans, and the MRI analyzed data includes T1-weighted and T2-weighted analyzed data derived from the MRI data from the T1-weighted and T2-weighted scans, and
wherein the inflammation assessment unit determines whether the inflammation of the gum tissue is acute or chronic from the T1-weighted and T2-weighted analyzed data.

7. An apparatus according to claim 4, wherein the at least one assessment unit further comprises a teeth embedded assessment unit that determines, from the analyzed MRI data, distances from areas where the teeth enter the bone to bottoms of roots of the teeth in the bone.

8. An apparatus according to claim 1, further comprising a user interface including a display device configured to display the assessment output by the output unit.

9. An apparatus according to claim 8, wherein the display device is configured to render an image generated from the MRI data.

10. An apparatus according to claim 1, further comprising an assessment scoring unit that determines a score indicative of the aspect of periodontal health of the individual based on the assessment of the analyzed MRI data by the at least one assessment unit.

11. An apparatus according to claim 10, wherein the score is determined based on a comparison of the assessment by the at least one assessment unit and a previous assessment of the individual.

12. An apparatus according to claim 10, wherein the score is in the form of at least one of a number that is relative to a given number scale and a color that is relative to a given color scale.

13. A system comprising:
the apparatus according to claim 1; and
an MRI scanning machine operatively linked to the apparatus such that MRI data from the MRI scanning machine is transmitted to the apparatus.

14. A method of assessing an aspect of the periodontal health of an individual, the method comprising:
using an MRI scanning machine to scan an oral area of an individual to thereby generate MRI data, the oral area including at least part of teeth, gum tissue, and a bone of the individual;
analyzing the MRI data to thereby generate analyzed MRI data in which at least one of hard tissue, soft tissue, an anatomical abnormality, a transition area between two tissues, or a transition area between tissue and an anatomical abnormality is identified, the analysis being performed by a computer; and
providing an assessment of periodontal heath of the individual based on the analyzed MRI data, the assessment being provided by the computer.

15. A method according to claim 14, wherein the assessment is at least one of (i) depths of dental pockets that extend from areas at a top of the gum tissue to areas where the teeth enter the bone, (ii) inflammation of the gum tissue, and (iii) distances from areas where the teeth enter the bone to bottoms of roots of the teeth in the bone.

16. A method according to claim 14, further comprising determining an assessment score indicative of the aspect of periodontal health based on the assessment of the analyzed MRI data.

17. A method according to claim 16, wherein the score is determined based on a comparison of the assessment and a previous assessment of the individual.

18. A method according to claim 16, wherein the score is in the form of at least one of a number that is relative to a given number scale and a color that is relative to a given color scale.

19. A method according to claim 14, wherein the MRI data is analyzed using quantitative mapping to thereby provide the assessment as inflammation of gum tissue.

20. A method according to claim 14, further comprising displaying on a display device an image rendered from the MRI data.

21. A method according to claim 14, wherein an MRI contrast agent is used in conjunction with the scan.

22. A method according to claim 14, wherein an intra-oral radiofrequency coil is used in conjunction with the scan.

23. A method according to claim 14, wherein a longitudinal assessment of inflammation scores or pocket depth determinations are provided.

24. A method according to claim 14, where anatomical information is derived from the analyzed MRI data, with the anatomical information identifying a long axis of a distinct tooth, and wherein a distance from an area where the distinct tooth enters the bone to a bottom of a root of the distinct tooth is assessed by evaluating signal variations around the distinct tooth as a function of position along the long axis of the distinct tooth.

25. A non-transitory computer readable storage medium storing one or more programs that when executed by a computer cause the computer to function as:
an MRI data receiving unit for receiving MRI data that was generated using an MRI scanning machine, the MRI data having been generated from a scan of an oral area of the individual, and the oral area including at least part of teeth, gum tissue, and a bone of the individual;
an MRI analysis unit for generating analyzed MRI data in which the at least one of hard tissue, soft tissue, an anatomical abnormality, a transition between two tissues, and a transition area between tissue and an anatomical abnormality is identified; and
at least one assessment unit for generating an assessment relating to an aspect of periodontal health of the individual based on the analyzed MRI data; and
an output unit for outputting the assessment generated by the at least one assessment unit.

26. The non-transitory computer readable storage medium according to claim 25, wherein the at least one assessment unit includes (i) pocket depth assessment unit that determines, from the analyzed MRI data, depths of dental pockets that extend from areas at a top of the gum tissue to areas where the teeth enter the bone, and (ii) an inflammation assessment unit that determines, from the analyzed MRI data, inflammation of the gum tissue.
